(19) United States
(12) Patent Application Publication
(10) Pub. No.: US 2016/0250027 A1
(43) Pub. Date: Sep. 1, 2016

(54) SYSTEM AND METHOD FOR IMPROVED CONSTRAINED PROSTHETIC ACETABULUM

(71) Applicant: The Curators of the University of Missouri, Columbia, MO (US)

(72) Inventors: Sonny Bal, Columbia, MO (US); Mohamed N. Rahaman, Rolla, MO (US)

(21) Appl. No.: 15/055,890

(22) Filed: Feb. 29, 2016

Related U.S. Application Data
(60) Provisional application No. 62/126,074, filed on Feb. 27, 2015.

(51) Int. Cl. A61F 2/34; A61F 2/36

(52) U.S. Cl. CPC A61F 2/34; A61F 2/3609; A61F 2002/347; A61F 2002/3208

(57) ABSTRACT

The invention provides an apparatus comprising a prosthetic femoral head and a liner. The prosthetic femoral head has a truncated spherical body, a truncated surface, and a stem cavity. The liner has a liner cavity and a rim wherein said liner cavity comprises a greater-than-hemispherical concavity and said rim comprises at least one slot. The slot allows insertion of the prosthetic femoral head into the liner cavity at an insertion orientation and retaining the prosthetic femoral head in the liner cavity at orientations other than the insertion orientation.

### CROSS-REFERENCE TO RELATED APPLICATIONS

This patent application claims priority to pending U.S. Provisional Patent Application Ser. No. 62/126,074, filed Feb. 27, 2015, and entitled “Constrained Acetabular Component,” the entire disclosure of which is incorporated herein by reference in its entirety.

### STATEMENT REGARDING FEDERALLY SPONSORED RESEARCH OR DEVELOPMENT

None.

### FIELD OF THE INVENTION

This invention relates to the field of prosthetic implants, more specifically, to an artificial hip joint with a constrained head and liner design (a constrained prosthetic acetabulum).

### BACKGROUND

During a total hip arthroplasty (THA), an anatomic acetabulum is reamed to remove diseased cartilage and expose healthy bleeding bone to accept the implantation of a prosthetic acetabular metal shell. A shell typically of hemispherical shape functions to engage a separate bearing or liner constructed of a material with low coefficient of friction such as polyethylene. The liner, in turn, mates or articulates with the newly implanted prosthetic femoral head or ball. In this manner the prosthetic implants form a mechanical head and liner joint similar to the original anatomical hip joint.

For prosthetic head and stem implantation, an endosteal femoral canal is typically prepared during surgery in order to implant the desired size of femoral implant that will replace the original anatomical femoral head and neck. The new prosthetic head is fixed usually with a Morse taper connection to the femoral stem after the latter has been impacted into the intramedullary canal of the femur. After that, the head is re-located into the prosthetic liner. Both the liner and the prosthetic acetabulum are typically, though not indefinitely, of hemispherical shape with the metal shell component being anchored into the pelvis via screws, biomedical cement, or mechanical press fit and the liner component being anchored into the prosthetic shell via a custom locking mechanism or biological cement.

After proper implantation of prosthetic devices, the head is physically reduced, i.e., positioned into the liner. It is important that the prosthetic equipment maintains the original placement of the head within the liner to avoid dislocation or subluxation of the joint. Normally, proper implant positioning and balancing of tissues during surgery and physiologic tension in the surrounding muscles serve to keep the head within the liner, i.e., prevent a hip dislocation. Equally important is the proper placement of the shell and liner in respect to the location of the original anatomical acetabulum to insure stability of the joint.

The most common liner design is of hemispherical shape or eccentric hemispherical shape. However, this design has no inherent constraint against dislocation built into the implant itself. The stability of hemispherical liners relies solely on the patient's muscles, tendons, and ligaments for prevention of dislocation. Many prosthetic total hip designs have attempted to increase the stability of the prosthetic joint and decrease the likelihood of dislocations. However, these designs are complex and require high manufacturing costs and additional time for surgery due to their use of an extra constraining component (e.g., locking ring) to prevent a femoral head from dislocation.

The present invention is directed to overcoming one or more of the problems set forth above.

### SUMMARY OF THE INVENTION

In one aspect of the invention, an apparatus for treating a total hip arthroplasty (THA), said apparatus comprising a prosthetic femoral head having a truncated spherical body, a truncated surface, and a stem cavity, wherein said truncated spherical body is greater than hemisphere; and a liner having a liner cavity and a rim, wherein said liner cavity comprises a greater-than-hemispherical concavity, wherein said rim comprises at least one slot, wherein said slot allows insertion of said prosthetic femoral head into said liner cavity at an insertion orientation and retention of said prosthetic femoral head in said liner cavity at orientations other than said insertion orientation.

In another aspect of the invention, a method for treating a total hip arthroplasty (THA), said method comprising: providing, a prosthetic femoral head having a truncated spherical body, a truncated surface, and a stem cavity, wherein said truncated spherical body is greater than hemisphere, a liner having a liner cavity and a rim, wherein said liner cavity comprises a greater-than-hemispherical concavity, wherein said rim comprises at least one slot; and inserting said prosthetic femoral head into said liner cavity at an insertion orientation wherein said slot allows said liner cavity to retain said prosthetic femoral head in said liner cavity at orientations other than said insertion orientation.

These are merely some of the innumerable aspects of the present invention and should not be deemed an all-inclusive listing of the innumerable aspects associated with the present invention. These and other aspects will become apparent to those skilled in the art in light of the following disclosure and accompanying drawings.

### BRIEF DESCRIPTION OF THE DRAWINGS

For a better understanding of the present invention, reference may be made to the accompanying drawings in which:

FIG. 1(a) illustrates a cross-sectional view of an exemplary embodiment of an apparatus for treating a total hip arthroplasty (THA);

FIG. 1(b) illustrates a 3D exploded view of the exemplary embodiment of the apparatus of FIG. 1(a);

FIG. 2(a) is a side sectional view of the liner according to the exemplary embodiment of the apparatus of FIG. 1(b);

FIG. 2(b) is a top sectional view of the liner according to the exemplary embodiment of the apparatus of FIG. 1(b);

FIG. 3(a) illustrates a first cross-sectional view of assembly process of the apparatus of FIG. 1(a);

FIG. 3(b) illustrates a second cross-sectional view of assembly process of the apparatus of FIG. 1(a);

FIG. 3(c) illustrates a third cross-sectional view of assembly process of the apparatus of FIG. 1(a);

FIG. 3(d) illustrates a fourth cross-sectional view of assembly process of the apparatus of FIG. 1(a);

FIG. 4(a) illustrates a first 3D exploded view of assembly process of the apparatus of FIG. 1(a);

FIG. 4(b) illustrates a second 3D exploded view of assembly process of the apparatus of FIG. 1(a);

FIG. 4(c) illustrates a third 3D exploded view of assembly process of the apparatus of FIG. 1(a);

FIG. 4(d) illustrates a fourth 3D exploded view of assembly process of the apparatus of FIG. 1(a);

FIG. 5(a) illustrates a cross-sectional view of the rotation range of the prosthetic femoral head when inserted into the liner according to the exemplary embodiment of the apparatus of FIG. 1(a);

FIG. 5(b) illustrates a cross-sectional view of the cranio-caudal line of an acetabulum and impingement angle;

FIG. 5(c) illustrates a cross-sectional view of spherical and trapezoidal femoral stem shafts with increased range of motion;

FIG. 6(a) illustrates a cross-sectional view of the apparatus of FIG. 1(a) in a two-component design;

FIG. 6(b) illustrates a cross-sectional view of the apparatus of FIG. 1(a) implemented in a three-component design;

FIG. 6(c) illustrates a cross-sectional view of the apparatus of FIG. 1(a) implemented in a four-component design;

FIG. 7(a) illustrates a cross-sectional view of the prosthetic femoral head inserted into the liner cavity before rotation according to the exemplary embodiment of the apparatus of FIG. 1(a);

FIG. 7(b) illustrates a cross-sectional view of the prosthetic femoral head after rotation according to the exemplary embodiment of the apparatus of FIG. 1(a); and

FIG. 7(c) illustrates a cross-sectional view of the combined acetabular prosthesis according to the exemplary embodiment of the apparatus of FIG. 1(a).

Reference characters in the written specification indicate corresponding items shown throughout the drawing figures.

### DETAILED DESCRIPTION OF THE INVENTION

Certain embodiments of the invention provide an improved constrained prosthetic acetabulum that maintains range of motion without component impingement and prevents dislocation. The improved constrained prosthetic acetabulum is easy to assemble, amenable to manufacture, resilient to component failure, and uniformly distributes forces during physiological movement and loading. An exemplary embodiment of the improved prosthetic acetabulum comprises a prosthetic femoral having with a truncated spherical body, a truncated surface, and a stem cavity wherein the truncated spherical body is greater than hemisphere. The exemplary embodiment also comprises a liner having a liner cavity and a rim wherein the liner cavity comprises a greater-than-hemispherical concavity and the rim comprises at least one slot. The slot allows insertion of the prosthetic femoral head into the liner cavity at an insertion orientation only and retains the prosthetic femoral head in the liner cavity at other orientations.

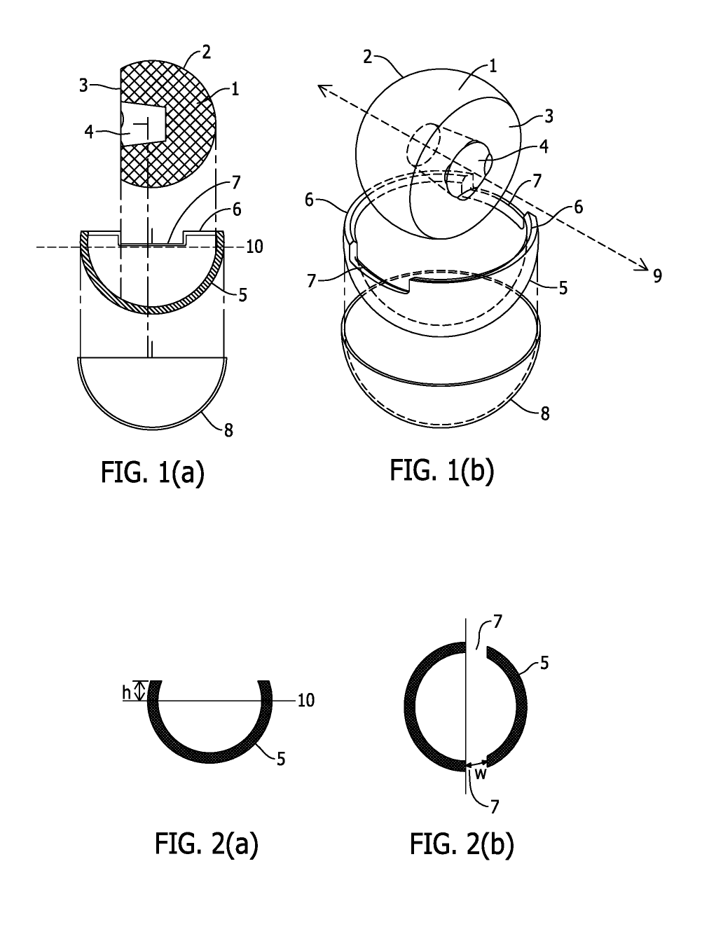

FIG. 1(a) illustrates a cross-sectional view of an exemplary embodiment of an apparatus for treating a total hip arthroplasty (THA) and FIG. 1(b) illustrates a 3D exploded view of the exemplary embodiment of the apparatus of FIG. 1(a). As shown in FIGS. 1(a) and 1(b), the exemplary embodiment comprises of a prosthetic femoral head 1, a liner 5, and a shell 8. The prosthetic femoral head 1 is configured to have a truncated spherical body 2, a truncated surface 3, and a stem cavity 4. The stem cavity is configured to penetrate the truncated spherical body 2 along a center axis 9 of the prosthetic femoral head 1. The liner 5 is configured to have a liner cavity and a rim 6. The liner cavity comprises a greater-than-hemispherical concavity as shown in FIG. 2(a). FIG. 2(a) is a side sectional view of the liner 5 of FIG. 1(b). In the exemplary embodiment, the height (h) of the liner 5 above the equatorial line 10 preferably ranges between 1/1,000 and ⅓ of the diameter of the liner 5 as shown in FIG. 2(a). The liner cavity comprises at least one slot 7. In the exemplary embodiment in which two slots are employed, the slots 7 are preferably non-diametrical to each other as shown in FIG. 2(b). FIG. 2(b) is a top sectional view of the liner 5 of FIG. 1(b). However, it should be understood that the slots 7 can also be placed diametrical to each other in other instances. Although only two slots 7 are shown in FIG. 1(b), single-slot or multiple-slots embodiments can also be implemented. The depth of the slot(s) can be preferably configured to extend to an equatorial line 10 as shown in FIG. 1(a). However, it should be understood that the depth of the slot can be set differently in other instances, for example, above or below the equatorial line 10, as long as the slot 7 allows the prosthetic femoral head 1 to be inserted into the liner cavity at an insertion orientation as explained further below. The width (w) of the slot 7 preferably ranges between 1/1,000 and ½ of the diameter of the liner 5 as shown in FIG. 2(b). The shell 8 is configured to have a shell cavity. The shell cavity 8 comprises a hemispherical concavity compatible to hosting the liner 5, as shown in FIGS. 1(a) and 1(b).

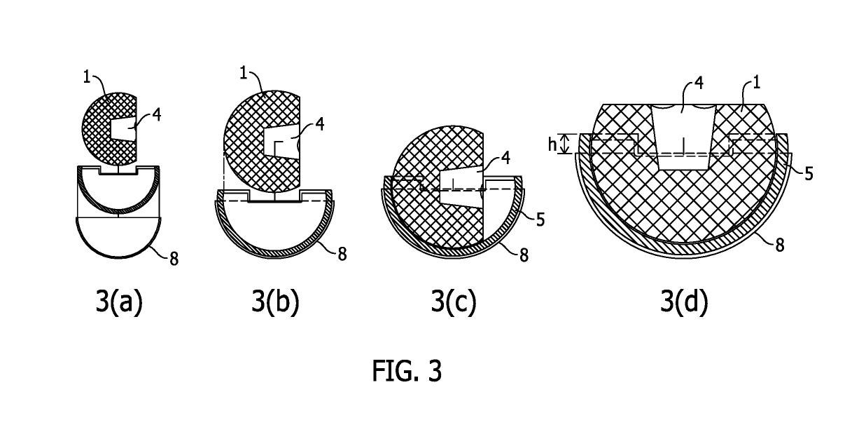

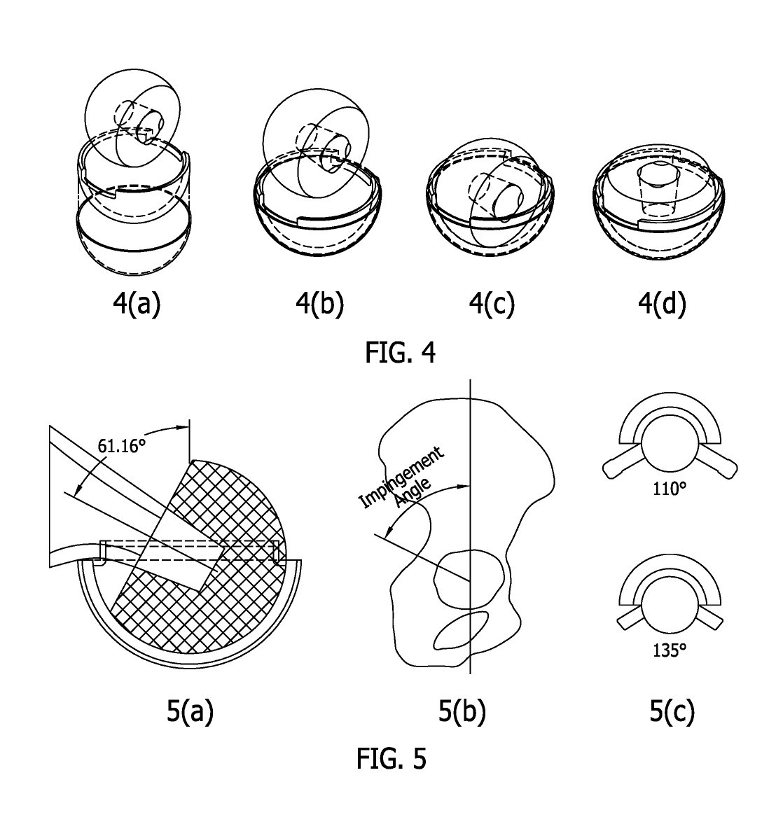

FIGS. 3(a) to 3(d) is a series of cross-sectional views of assembly process of the apparatus of FIG. 1(a). FIGS. 4(a) to 4(d) is a series of 3D exploded views of assembly process of the apparatus of FIG. 1(a). As shown in the schematic illustrations, the liner 5 with a slotted rim 6 contains precisely polished inner cavity surfaces such that an entirely spherical head, less the aforementioned truncated surface, can enter the liner cavity when rotated to an insertion orientation and translated in a direction normal to the bisecting cutting plane of the liner 5 that creates the opening of the liner cavity. As shown in FIGS. 3(a) to 3(b) and FIGS. 4(a) to 4(b), the prosthetic femoral head 1 is arranged to the insertion orientation: the prosthetic femoral head 1 is arranged such that the truncated surface 3 is aligned with one of the vertical edges of the slots 7 on the rim 6 of the liner 5. The polished inner surfaces on the liner 5 allow for the prosthetic femoral head 1 to enter the cavity of the liner 5 in an eccentric manner.

As shown in FIG. 3(c) and FIG. 4(c), after the prosthetic femoral head 1 enters the liner cavity and contacts the inner spherical surface of the liner 5, the prosthetic femoral head 1 can be rotated away from the insertion orientation. Due to the geometric design of the liner 5 and head 1, the spherical surfaces of the prosthetic femoral head 1 and liner cavity are mated into concentricity upon rotation of the prosthetic femoral head 1. The newly formed concentric relation between the internal cavity of the liner 5 and the spherical surface of the prosthetic femoral head 1 allows for containment/retention of the prosthetic femoral head 1 by the two spherical, or non-spherical, rim extensions on the liner 5, and by rim extensions elsewhere on the liner 5 that extend beyond the equatorial line 10, shown in FIG. 2(a).

Similarly, to remove the prosthetic femoral head 1 from the liner cavity, the prosthetic femoral head 1 can be rotated to the insertion orientation, perturbed in a direction to nullify the concentric relation between the spherical surface of the prosthetic femoral head 1 and the cavity of the liner 5, and translated out of the line cavity along the direction normal to the cutting plane that bisects the liner 5 and creates the opening cavity to the liner 5. As it is typically desired to maintain the prosthetic femoral head 1 within the cavity of the liner 5, the prosthetic femoral head 1 is preferably configured such that insertion of the femoral stem into the stem cavity of the prosthetic femoral head 1 physically inhibits rotation of the prosthetic femoral head 1 to the insertion orientation, a step that is required for removal of the prosthetic femoral head 1 from the liner 5.

The liner cut-out (e.g., the slot 7) that reduces the cut-out portion of the liner 5 combined with a more-than-hemispherical shape of the remaining liner 5 that allow the insertion of the prosthetic femoral head 1 into the liner 5 in one position only (e.g., insertion orientation). Other positions inherently lock the prosthetic femoral head 1 in the liner 5, while the range of movement and load distribution that are superior to other head-liner designs used in hip replacement devices are allowed. Such aspect of the apparatus of FIG. 1(a) provides certain advantages over other known designs.

In addition to the uniform load distribution of a head-in-liner configuration, the apparatus of FIG. 1(a) also allows for increased range of motion (ROM) since there are no extra constraining components (e.g., locking ring) that can lead to edge-impingement. Due to the ease of construction and manufacturing, both the liner 5 and the prosthetic femoral head 1 can be made of a plurality of materials such as highly cross-linked ultra-high molecular weight polyethylene (UHMWPE), cobalt-chrome alloys, and ceramics, all of which have been shown to resist wear and degradation related to the high torque loads of larger femoral head diameters. Thus, the head diameter to stem shaft ratio can be optimized resulting in an increased range of motion and decreased likelihood of impingement.

FIG. 5(a) illustrates a cross-sectional view of the rotation range of the prosthetic femoral head 1 when inserted into the liner 5. FIG. 5(a) shows the increased range of motion accomplished with manufactured liner slots. FIG. 5(b) illustrates a cross-sectional view of the cranio-caudal line of an acetabulum and impingement angle. The cranio-caudal line of an acetabulum and impingement angle as defined by Yamaguchi et al., (The spatial location of impingement in total hip arthroplasty. J Arthroplasty, 2000. 15(3): p. 305-13) with the liner slots positioned to increase ROM at the proposed impingement angle. FIG. 5(c) illustrates a cross-sectional view of spherical and trapezoidal femoral stem shafts with increased range of motion. FIG. 5(c) shows the increased ROM capability by using stems of trapezoidal cross section.

The apparatus of FIG. 1(a) allows for further increase in ROM, which in turn, provides decreased likelihood of impingement through the design of the manufactured slots. The design of the slots allows for eccentric entrance of the prosthetic femoral head into the liner cavity and requires no rim extensions and extra constraining component (e.g., locking ring) that are used in other known designs. As shown in FIG. 5(a), the slots are capable of producing over 120 degrees of motion along planes parallel to the slot cut plane.

As illustrated in FIG. 5(b), the primary location of impingement is at 78Q±2QQ posterior from the directly cephalic point in the acetabulum on surfaces extended from the articulation surface.

The apparatus of FIG. 1(a) addresses the contradictions and variability of impingement angles by allowing for free radial orientation of the liner 5 during installation such that the manufactured slots 7 may provide increased ROM according to the final mounted position, if the slots 7 are dialed in those locations in the native acetabular socket where the risk of impingement is the greatest. The idea of orienting the slots 7 and using the slots 7 to improve ROM and avoid impingement with this particular design is another advantage of the apparatus.

The apparatus of FIG. 1(a) also increases ROM with the specific femoral head shaft design used. Although the shaft can be configured to have a plurality of shapes, a specific shape that is most commonly used is the one that keeps the shaft neck at the smallest diameter while maintaining structural stability. As shown in FIG. 5(c), the shaft neck is preferably of trapezoidal cross section due to the increased range of motion of trapezoidal cross sections as compared to other known designs. Although a plurality of shapes can also be used for the portion of the stem inserted into the prosthetic femoral head cavity, a Morse taper design without skirting is preferably used in the exemplary embodiment because the Morse taper design allows for a smaller diameter femoral neck.

With the aforementioned design characteristics combined, the likelihood of impingement will be reduced, as the diameter of the head to the diameter of the shaft ratio will increase. In addition to wear, impingement often leads to fragmentation of the liner or femoral head, introduction of wear debris into the joint, and further increase of wear rate. Furthermore, the increased loading of the acetabular component due to impingement has been related to liner dissociation, shell failure, and loosening between the bone prosthesis interface.

In one embodiment, the apparatus of FIG. 1(a) contains surfaces along the edges of the shell 8 and liner 5 that are manufactured such that the surfaces prevent interference catch points between the shell 8 and the femoral stem. The manufactured surfaces serve three functions in that they increase the ROM of the femoral stem, decrease the likelihood of impingement as a result of restriction of motion from interfering surfaces, and decrease the likelihood of failure by reducing stress concentrations associated with manufacturing of surfaces with acute angles.

In one embodiment, the apparatus of FIG. 1(a) involves the use of the liner 5 with an articulation surface offset-and-eccentric or offset-and-concentric to the containing shell's spherical surface. This design has been shown to restore femoral offset allowing for lower rates of polyethylene wear, decreased prevalence of impingement, increased abductor function, and increased ROM. It should be understood that the exemplary embodiment can be applied to a plurality of systems, including, but not limited to, two component systems, three component systems, and four component systems. Each acetabular liner in the system may be concentric, offset and eccentric, or offset and concentric with respect to the acetabular component. In addition to variation in the number of components in the system and the offset and centricity of the liners is the increase in articulation polarity associated with increasing the number of components in the system. In this fashion, benefits of multiple components and multi-mobility systems can be exploited including decreased torsional loading on the prosthetic femoral head and increased stability associated with larger head sizes.

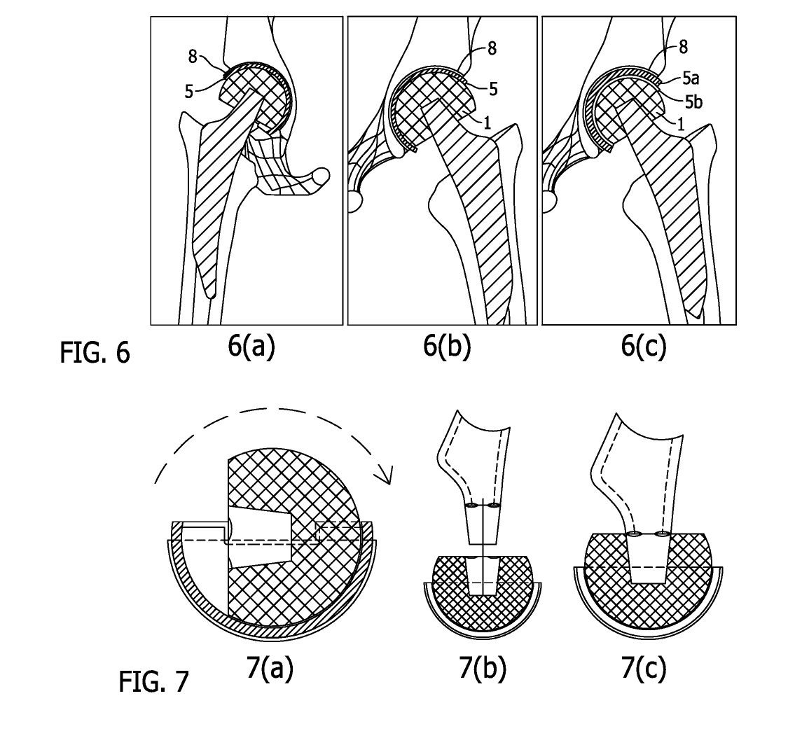

FIG. 6(a) illustrates a cross-sectional view of the apparatus of FIG. 1(a) in a two-component design. In FIG. 6(a), the acetabular shell 8 and liner 5 form a monoblock unit. FIG. 6(b) illustrates a cross-sectional view of the apparatus of FIG. 1(a) implemented in a three-component design. FIG. 6(c) illustrates a cross-sectional view of the apparatus of FIG. 1(a) implemented in a four-component design with both a first liner 5b and a first shell 5a. When used in the context of a two-component system, the primary components are (1) the femoral stem and head and (2) a monoblock acetabular liner and shell. Both the head component design and the acetabular design can be identical to the head design of the three component system illustrated in FIG. 6(b). The three-component system comprises the prosthetic femoral head 1 and stem, the liner 5, and the shell 8 as shown in FIG. 6(b). The head component takes a D-shaped profile when rotated to a specific angle and the acetabular component contains manufactured surfaces that allow for unique entry of the head into the acetabular cavity. The apparatus of FIG. 1(a) adds further simplicity in that the monoblock shell and liner allow decreased assembly time and decreased surgical time.

In the context of a four-component system, the primary components are a femoral stem and head, a first liner, a first shell with a cavity into which the first liner is inserted, and an acetabulum shell with a cavity into which the first shell and first liner are inserted. In this instance, the prosthetic femoral head and first liner design is similar to the previously defined head design of the three-component system, while the first shell design forms a shape similar to that of the previously defined shell design of the three-component design. The first shell and liner combination is then fit into the acetabular shell, which will have an internal surface with low coefficient of friction, thereby, allowing for the articulation between the first shell and liner combination within the acetabular shell. In a similar manner of expansion from a three-component system to a four-component system, the system is expandable to include a plurality of articulating surfaces and components.

In order for proper implementation of the invention in a surgical setting, a method of insertion is provided. First, a reaming process must commence in order for proper fixation of the prosthetic acetabular component to the host bony socket. After the reaming process and fixation of the acetabular component to the pelvis via screws, biomedical cement, or press fit, the liner 5 can be inserted into the prosthetic acetabulum. Similar to the acetabular prosthesis, the liner 5 can be affixed to the acetabular component via screws, biomedical cement, locking mechanism, or press fit. It should be noted that the liner 5 should be fit in a manner such that the manufactured slots 7 on the liner 5 increase ROM, thereby decreasing the likelihood of impingement.

After installation of the liner 5 in the shell 8, the prosthetic femoral head 1 can be inserted into the liner 5. For head insertion, the prosthetic femoral head 1 must first be rotated on its side such that the truncated surface 3 is aligned with the slots 7 of the liner 5, as shown in FIG. 3(b). This position is defined as the insertion orientation. Once the prosthetic femoral head 1 is in the proper insertion orientation, the prosthetic femoral head 1 can be translated along the axis orthogonal to the cutting plane bisecting the spherical liner and creating the opening cavity of the liner 5 until the prosthetic femoral head 1 is in contact with the liner 5. As previously explained, it is the precise design of the manufactured slots 7 as well as the polished surface of the prosthetic femoral head 1 that allows for positional eccentricity of the spherical surface of the head and the spherical surface of the liner cavity. This design facilitates entrance of the prosthetic femoral head 1 into the liner 5 cavity only at the insertion orientation during assembly.

FIG. 7(a) illustrates a cross-sectional view of the prosthetic femoral head 1 inserted into the liner cavity before rotation. In FIG. 7(a), the arrow above the image indicates one possible rotational motion for exposure of the stem cavity 4. FIG. 7(b) illustrates a cross-sectional view of the prosthetic femoral head 1 after rotation with the stem cavity 4 accessible for stem insertion. FIG. 7(c) illustrates a cross-sectional view of the combined acetabular prosthesis including the shell 8, liner 5, head 1, and stem. The exemplary embodiment shown in FIG. 7(c) shows the trapezoidal shape of the stem and the use of a Morse taper for press fit.

As shown in FIG. 7(a), the prosthetic femoral head 1 is rotated such that the manufactured surface of the prosthetic femoral head 1 is parallel to the bisecting plane of the liner 5 creating the opening of the liner cavity. The purpose of this rotation is two-fold: (1) forces the spherical surface of the prosthetic femoral head 1 into concentricity with the spherical surface of the liner cavity and (2) exposes the cavity within the prosthetic femoral head 1 allowing for the accessibility required for femoral stem insertion.

FIG. 7(b) shows a position in which the femoral stem can be inserted though the position for insertion. This position can be limited by the allowable ROM between the stem and the liner 5 defined after stem insertion. The stem can be affixed using one of threads, locking mechanism, biomedical cement, and press fit. The stem itself can comprise a plurality of shapes, although the stem is preferably characterized by a Morse taper press fitting with a trapezoidal cross-section along the shaft. The final insertion is shown in FIG. 7(c). It should be noted that the femoral reaming procedure has not been included herein. After the femur is machined for reception of the femoral stem, the femoral stem is placed and then inserted into the prosthetic femoral head.

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth.

1. An apparatus for treating a total hip arthroplasty (THA), said apparatus comprising:
a prosthetic femoral head having a truncated spherical body, a truncated surface, and a stem cavity, wherein said truncated spherical body is greater than hemisphere; and
a liner having a liner cavity and a rim, wherein said liner cavity comprises a greater-than-hemispherical concavity, wherein said rim comprises at least one slot, wherein said slot allows insertion of said prosthetic femoral head into said liner cavity at an insertion orientation and retention of said prosthetic femoral head in said liner cavity at orientations other than said insertion orientation.

2. The apparatus for a total hip arthroplasty (THA) according to claim 1, wherein a depth of said slot is extended to an equatorial line.

3. The apparatus for a total hip arthroplasty (THA) according to claim 1, wherein a width of said slot ranges between 1/1,000 and ½ of a diameter of said liner.

4. The apparatus for a total hip arthroplasty (THA) according to claim 1, wherein when two said slots are employed, said two slots are arranged diametrical to each other on said rim.

5. The apparatus for a total hip arthroplasty (THA) according to claim 1, wherein when two said slots are employed, said two slots are arranged non-diametrical to each other on said rim.

6. The apparatus for a total hip arthroplasty (THA) according to claim 1, wherein said truncated surface is configured to be aligned with one of vertical edges of said slot.

7. The apparatus for a total hip arthroplasty (THA) according to claim 1, wherein a height of said rim above an equatorial line ranges between 1/1,000 and ⅓ of a diameter of said liner.

8. The apparatus for a total hip arthroplasty (THA) according to claim 1, wherein said stem cavity penetrating said truncated spherical body along a center axis of said prosthetic femoral head.

9. The apparatus for a total hip arthroplasty (THA) according to claim 1, said apparatus further comprising:
a shell having a shell cavity, wherein said shell cavity comprises a hemispherical concavity compatible to said liner.

10. The apparatus for a total hip arthroplasty (THA) according to claim 1, said apparatus further comprising a femoral stem, wherein said femoral stem is configured to be inserted into said stem cavity of said prosthetic femoral head.

11. The apparatus for a total hip arthroplasty (THA) according to claim 10, wherein said femoral stem is comprised of a shaft neck having a trapezoidal cross-section.

12. A method for treating a total hip arthroplasty (THA), said method comprising:
providing,
a prosthetic femoral head having a truncated spherical body, a truncated surface, and a stem cavity, wherein said truncated spherical body is greater than hemisphere,
a liner having a liner cavity and a rim, wherein said liner cavity comprises a greater-than-hemispherical concavity, wherein said rim comprises at least one slot; and
inserting said prosthetic femoral into said liner cavity at an insertion orientation wherein said slot allows said liner cavity to retain said prosthetic femoral head in said liner cavity at orientations other than said insertion orientation.

13. The method for a total hip arthroplasty (THA) according to claim 12, wherein a depth of said slot is extended to an equatorial line.

14. The method for a total hip arthroplasty (THA) according to claim 12, wherein a width of said slot ranges between 1/1,000 and ½ of a diameter of said liner.

15. The method for a total hip arthroplasty (THA) according to claim 12, wherein when two said slots are employed, said two slots are arranged diametrical to each other on said rim.

16. The method for a total hip arthroplasty (THA) according to claim 12, wherein when two said slots are employed, said two slots are arranged non-diametrical to each other on said rim.

17. The method for a total hip arthroplasty (THA) according to claim 12, wherein said truncated surface is configured to be aligned with one of vertical edges of said slot.

18. The method for a total hip arthroplasty (THA) according to claim 12, wherein a height of said rim above an equatorial line ranges between 1/1,000 and ⅓ of a diameter of said liner.

19. The method for a total hip arthroplasty (THA) according to claim 12, wherein said stem cavity penetrating said truncated spherical body along a center axis of said prosthetic femoral head.

20. The method for a total hip arthroplasty (THA) according to claim 12, said method further comprising providing a shell having a shell cavity, wherein said shell cavity comprises a hemispherical concavity compatible to said liner.

21. The method for a total hip arthroplasty (THA) according to claim 12, said method further comprising providing a femoral stem, wherein said femoral stem is configured to be inserted into said stem cavity of said prosthetic femoral head.

22. The method for a total hip arthroplasty (THA) according to claim 21, wherein said femoral stem is comprised of a shaft neck having a trapezoidal cross-section.
